(19) United States
(12) Patent Application Publication
(10) Pub. No.: US 2016/0297779 A1
(43) Pub. Date: Oct. 13, 2016

(54) MODIFIED OXAZINE RESIN AND USE OF A COMPOSITION COMPRISING THE SAME

(71) Applicant: Elite Material Co., Ltd., Taoyuan City (TW)

(72) Inventors: Chen-Yu HSIEH, Taoyuan City (TW); Hui-Ting SHIH, Taoyuan City (TW)

(21) Appl. No.: 15/072,416

(22) Filed: Mar. 17, 2016

(30) Foreign Application Priority Data
Apr. 10, 2015 (TW) 104111625

(51) Int. Cl. C07D 265/12; C08F 222/08; C08G 73/06; H05K 1/03; C08K 5/5399; C08K 5/03; C08K 5/5313; C08J 5/24; C07D 265/16; C08K 3/36

(52) U.S. Cl. CPC C07D 265/12; C07D 265/16; C08F 222/08; C08G 73/06; C08K 3/36; C08K 5/5399; C08K 5/03; C08K 5/5313; C08J 5/24; H05K 1/0373; C08J 2335/06; C08J 2379/04

(57) ABSTRACT

The present disclosure is to provide a modified oxazine compound and use of a composition comprising the same.

### TECHNICAL FIELD

The present disclosure relates to a modified oxazine compound and a method of producing the same, in particular, to a modified oxazine compound for a copper clad laminate and a printed circuit board.

### BACKGROUND ART

Recently, with the rapid progress of electronic technologies, the information processing of electronics such as those for mobile communication, servers, cloud storages has been developed toward “high-frequency and high-speed digitization of signal transmission”. Thus, in the field of the laminate with high transmission rate, a resin with low dielectric properties has been the main research interest to meet the requirement of fast information processing for cloud technology or terminal server. The requirement for the laminate such as copper clad laminate (CCL) includes that the laminate should be made from the material having high reliability, high heat and moisture resistance, low dielectric constant, low dissipation factor, high dimension stability, and the like. Accordingly, there is a need for a material for high performance copper clad laminate having superior dielectric properties to produce a high performance printed circuit board (PCB).

Benzoxazine compounds have advantages such as excellent heat resistance and mechanical properties. Taiwan Patent No. 308566 discloses a resin composition for producing a laminate, wherein the resin composition is formed from a benzoxazine compound and a thermosetting resin. Taiwan Patent No. 460537 discloses a composition for producing a laminate, wherien the composition is formed from a benzoxazine compound and a phenol novolac resin. Taiwan Patent No. 583258 discloses a composition for producing a laminate, wherein the composition is formed from a benzoxazine compound and a triazine novolac resin. Taiwan Patent No. 1311568 discloses the use of a benzoxazine compound and a styrene-maleic anhydride copolymer in making a laminate. The conventional benzoxazine compounds (such as bisphenol A type benzoxazine and bisphenol F type benzoxazine), however, still have the following disadvantages: relatively low glass transition temperature (Tg) and relatively poor dielectric properties, and thus failure to satisfy the requirement of lower dielectric properties and of higher glass transition temperature in a new generation high performance product. Accordingly, there is a need to propose a benzoxazine compound having relatively high glass transition temperature and relatively preferred dielectric properties so as to produce a laminate useful as a desirable material for a PCB at high frequency/high transmission rate.

### SUMMARY OF INVENTION

For the present technical problems described above, the present disclosure provides a modified oxazine compound for use in a resin composition. The resin composition may be used in producing a prepreg or a resin film. The copper clad laminate or the printed circuit board made from the prepreg or the resin film has characteristics such as low dielectric constant, low dissipation factor, high temperature resistance, and high flame retardant.

To achieve the aforesaid purpose, the present disclosure provides a modified oxazine compound having a structure represented by the following formula (1) or formula (2):

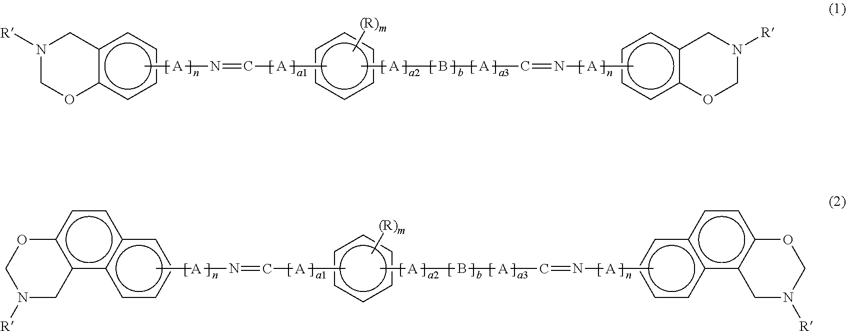

wherein R may be an aliphatic hydrocarbyl group (such as an alkyl group, a cycloalkyl group, an alkenyl group) or an aryl group (such as phenyl, benzyl); R′ is selected from the group consisting of imino, allyl, a C₁-C₂₀ (i.e. 1 to 20 carbon atoms) aliphatic hydrocarbyl group (such as an alkyl group, a cycloalkyl group, an alkenyl group), dicyclopentadienyl, and an aryl group (such as phenyl, benzyl); wherein allyl, C₁-C₈ alkyl group, C₃-C₈ cycloalkyl group, phenyl or benzyl are preferred, and R′ can be further substituted with 1 to 4 substituents. m may represent an integer of 0 to 4. n may be 0 or 1. A may be selected from the group consisting of —CH₂—, —CH(CH₃)—, and —C(CH₃)₂— and is the same or different at each occurrence. B is an arylene group (such as phenylene, benzylene). Further, B is a substituted arylene group (such as brominated phenylene, brominated benzylene). a1, a2, a3 and b may be each independently 0 or 1.

In an embodiment, the modified oxazine compound is selected from the group consisting of benzoxazine and naphth-oxazene. In an embodiment, the modified oxazine compound of the present disclosure has a structure represented by formula (1), wherein a1=0, a2=0, a3=0, b=0, m=0 and n=0, and R′ is phenyl. In another embodiment, the modified oxazine compound of the present disclosure has a structure represented by formula (2), wherein a1=0, a2=0, a3=0, b=0, m=0 and n=0, and R′ is phenyl. In a further embodiment, the modified oxazine compound of the present disclosure has a structure respectively represented by formula (1) and formula (2), wherein a1=0, a2=0, a3=0, b=1, m=0 and n=0, B is phenylene (—C₆H₄—), and R′ is phenyl. In yet another embodiment, the modified oxazine compound of the present disclosure has a structure represented by formula (1) or formula (2), wherein a1=0, a2=1, a3=0, b=1, m=0 and n=0, and A is —C(CH₃)₂—, B is phenylene (—C₆H₄—), and R′ is phenyl. In still another embodiment, the modified oxazine compound of the present disclosure has a structure represented by formula (1) or formula (2), wherein a1=1, a2=0, a3=1, b=1, m=0 and n=0, and A is methylene (—CH₂—), B is phenylene (—C₆H₄—), and R′ is phenyl.

In preferred embodiments, the modified oxazine compound of the present disclosure is the compound having a structure selected from the group consisting of the following formula (6), formula (7), formula (8), formula (9), and formula (10):

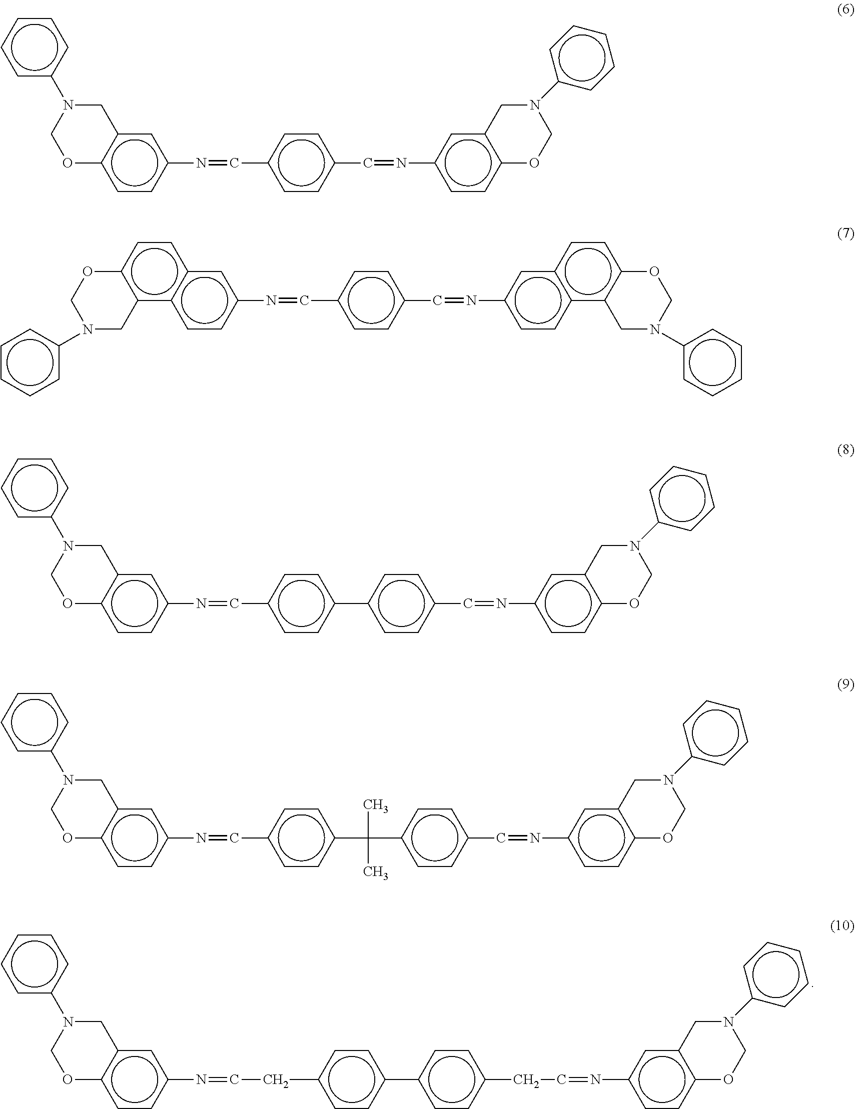

The present disclosure further provides a method of producing a modified oxazine compound comprising: adding a phthalaldehyde compound and an aminophenol compound into a solvent followed by reacting the mixture at 100 to 150° C. for 3 to 5 hours to form an azomethine group-containing phenol compound followed by reacting the resulting azomethine group-containing phenol compound with a primary amine and formaldehyde at 70 to 100° C. for 5 to 8 hours to afford a modified oxazine compound.

In the aforesaid method, the phthalaldehyde compound has a structure represented by formula (3):

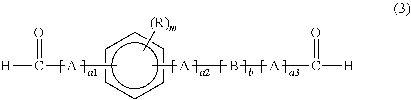

wherein R may be an aliphatic hydrocarbyl group (such as an alkyl group, a cycloalkyl group, an alkenyl group) or an aryl group (such as phenyl, benzyl). m may represent an integer of 0 to 4. A may be selected from the group consisting of —CH₂—, —CH(CH₃)—, and —C(CH₃)₂— and two A's may be the same or different; B may be an arylene group (such as phenylene, benzylene). Further, B is a substituted arylene group (such as brominated phenylene, brominated benzylene). a1 to a3 and b may be each independently 0 or 1.

For example, the phthalaldehyde compound may be o-phthalaldehyde, m-phthalaldehyde, p-phthalaldehyde, 4,6-dimethylisophthalic dialdehyde (CAS No.: 25445-41-4), 4-methylisophthalaldehyde (CAS No.: 23038-58-6), or 4,4′-biphenyldicarboxaldehyde (CAS No.: 66-98-8).

The preferred phthalaldehyde compound is selected from o-phthalaldehyde, m-phthalaldehyde, or p-phthalaldehyde.

In the aforesaid method, the aminophenol compound may be the compound having the structure represented by, but not limited to, the following formula (4) or formula (5):

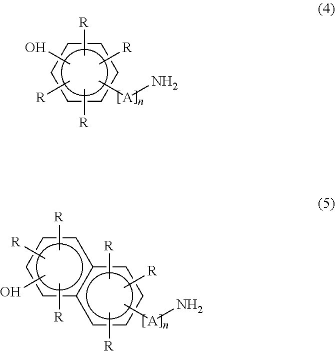

wherein R are each independently selected from hydrogen, an aliphatic hydrocarbyl group (such as an alkyl group, a cycloalkyl group, an alkenyl group) or an aryl group (such as phenyl, benzyl); A is selected from the group consisting of —CH₂—, —CH(CH₃)—, —C(CH₃)₂—, an aliphatic hydrocarbylene group, and an arylene group; and n may be 0 or 1.

Examples of the aminophenol compound include, but are not limited to 2-aminophenol, 3-aminophenol, 4-aminophenol, 2,4-diaminophenol (CAS No.: 95-86-3), 2,6-dichloro-p-aminophenol (CAS No.: 5930-28-9), 6-amino-2-naphthol (CAS No: 56961-71-8), or 8-amino-2-naphthol (CAS No: 118-46-7).

Preferred aminophenol compounds are selected from the group consisting of 2-aminophenol, 3-aminophenol, 4-aminophenol, 6-amino-2-naphthol, and 8-amino-2-naphthol.

In the aforesaid method, the primary amine is one having a structure represented by general formula R′NH₂, wherein R′ is selected from the group consisting of imino, allyl, a C₁-C₂₀ aliphatic functional group (such as an alkyl group, a cycloalkyl group, or an alkenyl group), dicyclopentadienyl, and an aryl group (such as phenyl, benzyl), wherein R′ is preferably allyl, a C₁-C₈ alkyl group, a C₃-C₈ cycloalkyl group, phenyl or benzyl. R′ can be further substituted with 1 to 4 substituents.

Examples of the primary amine compound include, but are not limited to aniline, o-phenylenediamine, m-phenylenediamine, p-phenylenediamine, biphenyldiamine, 4,4′-diaminodiphenylmethane, cyclohexylamine, butylamine, methylamine, hexylamine, allylamine (CAS No.: 107-11-9) or propanediamine.

Preferred primary amine compounds are selected from the group consisting of aniline, cyclohexylamine, butylamine, methylamine, hexylamine, and allylamine.

In the aforesaid method, the solvent is selected from any one of dimethyl sulfoxide, dimethylformamide, dimethylacetamide, toluene, and xylene, or a mixture thereof.

A modified oxazine can be obtained by conducting the aforesaid reaction sequence and conditions. For example, the obtained product may have a structure represented by above formula (6), formula (7), formula (8), formula (9), or formula (10). However, the modified oxazine of the present disclosure is not limited thereto.

When compared with conventional benzoxazine compounds, the modified oxazine compound of the present disclosure has at least the following advantages: low dissipation factor and high heat resistance (such as high glass transition temperature).

Another object of the present disclosure is to provide a resin composition with low dissipation factor, comprising: (A) a modified oxazine compound; and (B) a crosslinking agent.

The modified oxazine compound of the present disclosure can be a combination of the monomer thereof or a prepolymer thereof.

The crosslinking agent as described herein may be any one or a combination of: an epoxy resin, a cyanate resin, isocyanate, a polyphenylene ether resin, maleimide, a polyamide, a polyimide, a phenoxy resin, a styrene-maleic anhydride copolymer, a polyester, a polyolefin, a phenol resin, an amine-based curing agent, an anhydride-based curing agent, and diallyl bisphenol A.

The epoxy resin as described herein may be any one or a combination of: bisphenol A epoxy resin, bisphenol F epoxy resin, bisphenol S epoxy resin, bisphenol AD epoxy resin, phenolic novolac epoxy resin, bisphenol A novolac epoxy resin, bisphenol F novolac epoxy resin, o-cresol novolac epoxy resin, trifunctional epoxy resin, tetrafunctional epoxy resin, multifunctional epoxy resin, dicyclopentadiene (DCPD) type epoxy resin, phosphorous-containing epoxy resin, DOPO-containing epoxy resin, DOPO-HQ-containing epoxy resin, p-xylene epoxy resin, naphthalene-based epoxy resin, benzopyran-based epoxy resin, biphenyl novolac epoxy resin, isocyanate modified epoxy resin, phenol benzaldehyde epoxy resin, and phenol aralkyl novolac epoxy resin. DOPO-containing epoxy resin can be DOPO-containing phenolic novolac epoxy resin, DOPO-containing o-cresol novolac epoxy resin, or DOPO-containing bisphenol novolac epoxy resin. DOPO-HQ-containing epoxy resin can be DOPO-HQ-containing phenolic novolac epoxy resin, DOPO-HQ-containing o-cresol novolac epoxy resin, or DOPO-HQ-containing phenolic novolac epoxy resin.

Examples of the cyanate resin include, but are not limited to: a cyanate resin having the structure Ar—O—C≡N, wherein Ar may be a substituted or unsubstituted aryl group; novolac-based cyanate resin; bisphenol A type cyanate resin; bisphenol A novolac type cyanate resin; bisphenol F type cyanate resin; bisphenol F novolac type cyanate resin; a dicyclopentadiene-containing cyanate resin; a naphthalene ring-containing cyanate resin; or phenolphthalein type cyanate resin.

Examples of the cyanate resin include, but are not limited to: the cyanate resins under the trade name such as Primaset PT-15, PT-30S, PT-60S, CT-90, BADCY, BA-100-10T, BA-200, BA-230S, BA-300S, BTP-2500, BTP-6020S, DT-4000, DT-7000, Methylcy, ME-240S (all manufactured by Lonza), and the like.

The isocyanate as described herein includes, but is not limited to any one or a combination of: 1,4-cyclohexane diisocyanate, isophorone diisocyanate, methylene bis(4-cyclohexylisocyanate), triallyl isocyanurate, hydrogenated 1,3-xylylene diisocyanate, and hydrogenated 1,4-xylylene diisocyanate. Preferably, the isocyanate is triallyl isocyanurate.

The polyphenylene ether resin as described herein is preferably selected from the group consisting of at least one of the following: dihydroxyl polyphenylene ether (such as SA-90, available from Sabic), bisvinylbenzyl polyphenylene ether resin(such as OPE-2st, available from Mitsubishi Gas Chemical Co., Inc.), vinylbenzylated modified bisphenol A polyphenylene ether, methacrylate terminated polyphenylene ether (such as SA-9000, available from Sabic), and any combinations thereof, but is not limited thereto.

The maleimide as described herein includes, but is not limited to any one or a combination of: 4,4′-diphenylmethane bismaleimide, oligomer of phenylmethane maleimide, m-phenylene bismaleimide, bisphenol A diphenyl ether bismaleimide, 3,3′-dimethyl-5,5′-diethyl-4,4′-diphenylmethane bismaleimide, 4-methyl-1,3-phenylene bismaleimide, and 1,6-bismaleimide-(2,2,4-trimethyl hexane).

The phenoxy resin as described herein refers to those resins having phenoxy or its derivative group as the backbones. The phenoxy resin can be prepared through reacting a bisphenol compound or a derivative thereof with epichlorohydrin or a derivative thereof and then obtaining the product by conventional processes.

Examples of the phenoxy resin include, but are not limited to: E1255HX30 (bisphenol A skeleton), E1256B40 (bisphenol A skeleton), E4256H40 (bisphenol F skeleton), E5580BPX40, YX8100BH30, YL6954BH30, produced by Japan Epoxy Resins Co., Ltd.; ERF001, produced by Tohto Kasei Co., Ltd.; RX200, produced by Taiyo Ink Mfg. Co., Ltd.

For the styrene-maleic anhydride copolymer as described herein, the ratio of the styrene (S) to maleic anhydride (MA) (S/MA) can be 1/1, 2/1, 3/1, 4/1, 6/1 or 8/1, such as the styrene-maleic anhydride copolymers sold by Cray valley under the trade names SMA-1000, SMA-2000, SMA-3000, EF-30, EF-40, EF-60, EF-80, and the like. In addition, the styrene-maleic anhydride copolymer can be esterified styrene-maleic anhydride copolymer, such as those commercially available under the trade names SMA1440, SMA17352, SMA2625, SMA3840, and SMA31890. Any one of the aforesaid styrene-maleic anhydride copolymer products or a combination thereof can be used for addition to the resin composition of the present disclosure.

The polyester resin of the present disclosure is made by esterifying aromatics having dicarboxylic acid group with aromatics having dihydroxyl group, such as HPC-8000T65 available from Dainippon Ink and Chemicals, Inc.

The polyolefin as described herein may be any one or a combination of: styrene-butadiene-divinylbenzene terpolymer, styrene-butadiene-maleic anhydride terpolymer, vinyl-polybutadiene-urethane oligomer, styrene butadiene copolymer, hydrogenated styrene butadiene copolymer, styrene isoprene copolymer, and hydrogenated styrene isoprene copolymer.

The polyolefin is preferably selected from styrene-butadiene-divinylbenzene terpolymer, styrene-butadiene-maleic anhydride terpolymer, vinyl-polybutadiene-urethane oligomer, or a combination thereof.

The phenol resin as described herein can be a monofunctional, a bifunctional, or a multifunctional phenol resin. The type of the phenol resin used is not limited. All of the phenol resins used in the art can be adopted as the phenol resins herein.

The amine-based curing agent of the present disclosure is the resin having an amino group, preferably having two amino functional groups (diamino). Said curing agent is the same as hardener and crosslinking agent. Particularly, the amine-based curing agent can be one or a combination of diamino diphenyl sulfone, diamino diphenyl methane, diamino diphenyl ether, diamino diphenyl sulfide and dicyandiamide (DICY). Preferably, the amine-based curing agent is selected from one or a combination of 4,4′-diamino diphenyl sulfone; 4,4′-diamino diphenyl methane; 4,4′-diamino diphenyl ether; 4,4′-diamino diphenyl sulfide; and dicyandiamide (DICY).

The anhydride-based curing agent as described herein can be liquid, solid, or multifunctional. Said curing agent is the same as hardener and crosslinking agent. The type of the anhydride-based curing agent used is not limited. All of the anhydride-based curing agents used in the art can be adopted as the anhydride-based curing agents herein.

The resin composition as described herein can further comprise a modifier to adjust at least one of the following properties: flame retardancy, heat resistance, dielectric constant, dissipation factor, toughness, reactivity, viscosity, and solubility.

In one embodiment of the present disclosure, the modifier is selected from the group consisting of a flame retardant, a curing accelerator, an inorganic filler, a surfactant, a toughening agent, solvent, and the combination thereof.

The flame retardant as described herein can be a phosphorus-containing flame retardant or a brominated flame retardant, wherein the brominated flame retardant is not particularly limited and may be preferably at least one selected from the group consisting of ethylene-bis(tetrabromophthalimide) (such as SAYTEX BT-93, which is commercially available from Albemarle); ethane-1,2-bis(pentabromophenyl) (such as SAYTEX 8010, which is commercially available from Albemarle); and 2,4,6-Tris{2,4,6-tribromophenoxy)-1,3,5-triazine (such as the product produced by ICL Industrial under the trade name FR-245). The phosphorus-containing flame retardant is not limited and may be preferably at least one selected from the group consisting of: bisphenol A bis-(diphenylphosphate); ammonium polyphosphate; hydroquinone bis-(diphenyl phosphate); bisphenol A bis-(diphenylphosphate); tri(2-carboxyethyl)phosphine(TCEP); tris(chlo ro isopropyl) phosphate; trimethyl phosphate(TMP); dimethyl methylphosphonate(DMMP); resorcinol bis(dixylenyl phosphate)(RDXP), such as PX-200 (i.e. resorcinol bis(di-(2,6-xylenyl) phosphate)); pho sphazenes, such as SPB-100; melamine polypho sphate; 9,10-dihydro-9-oxa-10-phosphaphenanthrene-10-oxide (DOPO) and derivatives or resin thereof; melamine cyanurate; and tri-hydroxy ethyl isocyanurate. However, the flame retardant as described herein is not limited thereto. For example, the flame retardant may be a DOPO compound; a DOPO resin (such as DOPO-HQ, DOPO-NQ, DOPO-PN, DOPO-BPN); a DOPO-containing epoxy resin, and the like, wherein the DOPO-PN is a DOPO-containing phenolic novolac; and DOPO-BPN can be the DOPO-containing bisphenol novolac compound such as DOPO-BPAN (i.e. DOPO-containing bisphenol A novolac), DOPO-BPFN (i.e. DOPO-containing bisphenol F novolac), DOPO-BPSN (i.e. DOPO-containing bisphenol S novolac), and the like.

The resin composition as described herein can further comprise a curing accelerator to increase the reaction rate of the resin composition. The curing accelerator may comprise a catalyst such as a Lewis base or a Lewis acid, wherein the Lewis base may include one or more of imidazole, a boron trifluoride-amine complex, ethyl triphenyl phosphonium chloride, 2-methylimidazole (2MI), 2-phenyl-1H-imidazole (2PZ), 2-ethyl-4-methylimidazole (2E4MI), triphenylphosphine (TPP), and 4-dimethylaminopyridine (DMAP). The Lewis acid may include a metal salt such as manganese salt, iron salt, cobalt salt, nickel salt, copper salt, and zinc salt, e.g. the metal catalyst zinc isooctanoate, cobalt isooctanoate, and the like.

The resin composition of the present disclosure can further comprise an inorganic filler to increase the heat conductivity and to improve the properties such as thermal expansion, mechanical strength, and the like, of the resin composition. The inorganic filler is preferably evenly distributed in the resin composition. The inorganic filler may include silica (fused, non-fused, porous, or hollow), alumina, aluminum hydroxide, magnesia, magnesium hydroxide, calcium carbonate, aluminum nitride, boron nitride, aluminum silicon carbide, silicon carbide, titanium dioxide, zinc oxide, zirconia, mica, boehmite (AlOOH), calcined talc, talc, silicon nitride, and/or calcined kaolin. In addition, the shape of the inorganic filler can be sphere, fiber, plate, granule/particle, sheet or whisker and can be optionally pretreated with a silane coupling agent or a siloxane coupling agent. The inorganic filler can be a granular powder having a particle size of equal to or less than 100 μm, preferably 10 nm to 20 μm. The inorganic filler is most preferably a granular powder having a nanoscale particle size equal to or less than 1 μm.

The resin composition of the present disclosure can further comprise a surfactant to allow the inorganic filler evenly distributed in the resin composition. The surfactant can comprise silanes and/or siloxanes.

The resin composition of the present disclosure may further comprise a toughening agent to improve the toughness of the resin composition, wherein the toughening agent may comprise a rubber resin, carboxyl-terminated butadiene acrylonitrile (CTBN) rubber, core-shell polymer, or the like.

The resin composition of the present disclosure may further comprise a solvent to change the solid content of and to adjust the viscosity of the resin composition, wherein the solvent may comprise methanol, ethanol, ethylene glycol mono methylether, acetone, butanone (methyl ethyl ketone (MEK)), methyl isobutyl ketone, cyclohexanone, toluene, dimethylbenzene, methoxyethyl acetate, ethoxyethyl acetate, propoxyethyl acetate, ethyl acetate, dimethylformamide, propylene glycol methyl ether, or mixtures thereof.

The resin composition of the present disclosure may be further mixed with any one or a combination of the following benzoxazine resins: bisphenol A type benzoxazine resin, bisphenol F type benzoxazine resin or phenolphthalein type benzoxazine resin, dicyclopentadiene type benzoxazine resin, phosphorus-containing benzoxazine resin, such as the product under the trade name LZ-8270, LZ-8280, or LZ-8290, manufactured by Huntsman; or the product under the trade name HFB-2006M, manufactured by Showa Highpolymer Co., Ltd.

The present disclosure further provides a prepreg, which is made from the aforesaid resin composition, has the characteristics such as low dissipation factor and high heat resistance (such as high glass transition temperature). Accordingly, the prepreg disclosed herein may comprise a reinforcement material and the aforesaid resin composition, wherein the resin composition is attached to the reinforcement material by, for example, impregnation, and then is in a semi-cured state after heating (such as baking) at an elevated temperature. The reinforcement material can be a fiber material, woven fabric and non-woven fabric, such as fiberglass cloth, to increase the mechanical strength of the prepreg. In addition, the reinforcement material can be optionally pre-treated with a silane coupling agent.

The prepreg can be cured to form a cured sheet or cured insulating layer under high temperature heating (such as baking) or under high temperature and high pressure condition. If the resin composition contains solvent, the solvent will evaporate and thus be removed during the process of high-temperature heating.

Another object of the present disclosure is to provide a resin film which is made from the aforesaid resin composition, has the characteristics such as low dissipation factor and high heat resistance (such as high glass transition temperature). The resin film comprises the aforesaid resin composition. The resin film can be formed by coating the resin composition on a PET film (a polyester film) or on a PI film (a polyimide film) or on a copper foil (to obtain a resin-coated copper) followed by baking with heat.

Yet another object of the present disclosure is to provide a laminate, such as copper clad laminate, made from the aforesaid prepreg or resin film has the characteristics such as low dissipation factor and high heat resistance (such as high glass transition temperature) and is particularly suitable for use in the circuit board for high-speed and high-frequency signal transmission. Accordingly, the present disclosure provides a laminate comprising two or more metal foils and at least one insulating layer. The metal foil, such as copper foil, may further comprise at least one of aluminum alloy, nickel alloy, platinum alloy, silver alloy, and gold alloy. The insulating layer is formed by curing the aforesaid prepreg or resin film under high temperature and high pressure. For example, the insulating layer can be formed by stacking the aforesaid prepreg between two metal foils followed by laminating under high temperature and high pressure.

The laminate of the present disclosure has at least one of the following advantages: low dissipation factor and high heat resistance (such as high glass transition temperature). The laminate can be used to form a circuit board after further processing such as wiring. After bonding electronic elements to the circuit board, the quality of the resulting circuit board will not be influenced when operated under stringent environment such as high temperature and high humidity.

According, still another object of the present disclosure is to provide a printed circuit board which is made of the aforesaid laminate and has the characteristics such as low dissipation factor and high heat resistance (such as high glass transition temperature) and is suitable for high-speed and high-frequency signal transmission. The circuit board comprises at least one aforesaid laminate and can be made by any conventional processes.

### BRIEF DESCRIPTION OF DRAWING(S)

FIG. 1 shows a graph of change in enthalpy of the product Compound B measured by differential scanning calorimeter (DSC). X-axis represents the temperature (unit: ° C.), and y-axis represents the heat flow (unit: W/g).

FIG. 2 shows the result of Tg of the product Compound B measured by differential scanning calorimeter (DSC). X-axis represents the temperature (unit: ° C.), and y-axis represents the heat flow (unit: W/g).

FIG. 3 is FTIR (fourier transform infrared spectroscopy) spectrum of the reaction precursor Compound A. X-axis represents the wavenumber (unit: cm⁻¹), and y-axis represents the transmittance (unit: T %).

FIG. 4 is FTIR spectrum of the product Compound B. X-axis represents the wavenumber (unit: cm⁻¹), and y-axis represents the transmittance (unit: T %).

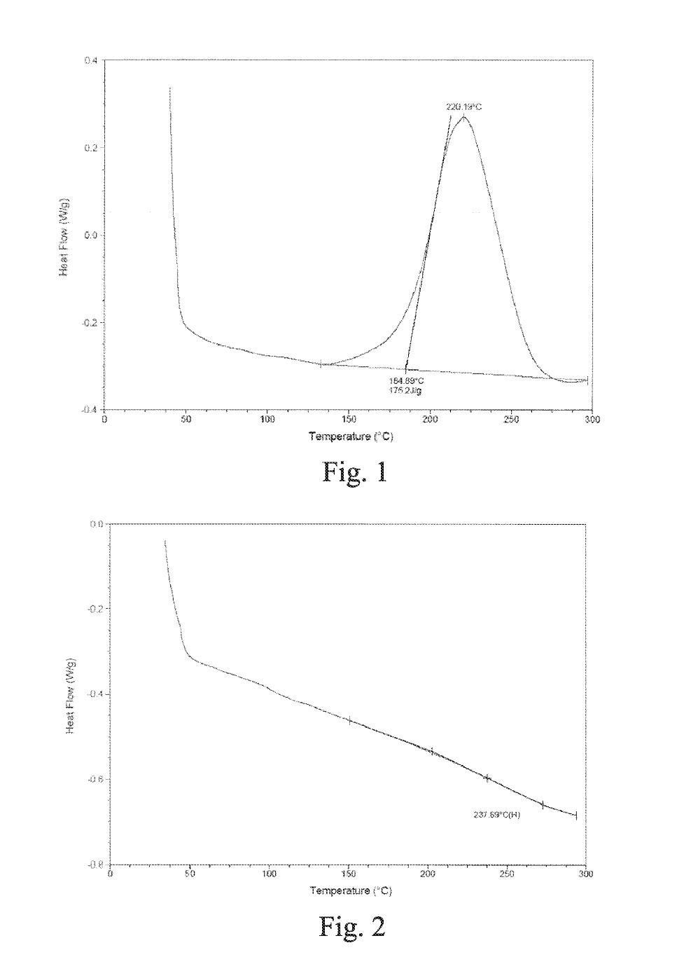

### DESCRIPTION OF EMBODIMENTS

To further disclose the invention so that the objects, features and advantageous effects of the invention will be apparent to those having ordinary skill in the art and that the invention may be carried out, several examples with reference to the accompanying drawings will be provided to further explain the invention. However, it should be noted that the following examples are intended to further explain the invention and should not be construed as a limitation on the actual applicable scope of the invention, and as such, all modifications and alterations without departing from the spirits of the invention shall remain within the protected scope and claims of the invention.

### PRODUCTION EXAMPLE

3 L reaction vessel equipped with a reflux condenser, a thermometer, and a stirring device was set up. 134.0 grams (g) (1 mol) of p-phthalaldehyde, 218 g (2 mol) of 4-aminophenol, 205.7 g of propylene glycol monomethyl ether, and 178.0 g of toluene were added to the reaction vessel to form a mixture. The mixture was stirred, heated, followed by 4-hour refluxing with removal of the produced water at 115 to 125° C. After cooling to room temperature, a polyazomethine compound (Compound A) was yielded. 388 g of Compound A and then 172 g of formaldehyde, 242 ml of xylene and 484 ml of butanol were added into a 3 L glass jacketed reaction vessel to form a reaction mixture. The reaction mixture was heated to between 80° C. and 82° C. and then continuously stirred. Finally, 238 g of aniline was added. The mixture was heated to between 90° C. and 95° C. and then was refluxed for 6 hours. The resulting reaction mixture was added with additional 600 ml of xylene and 1200 ml of butanol to cool the reaction temperature to room temperature. After removal of the alcohol solvent, the modified benzoxazine compound (abbreviated as modified Bz or Compound B hereinafter) product with a solids content of 70% was yielded.

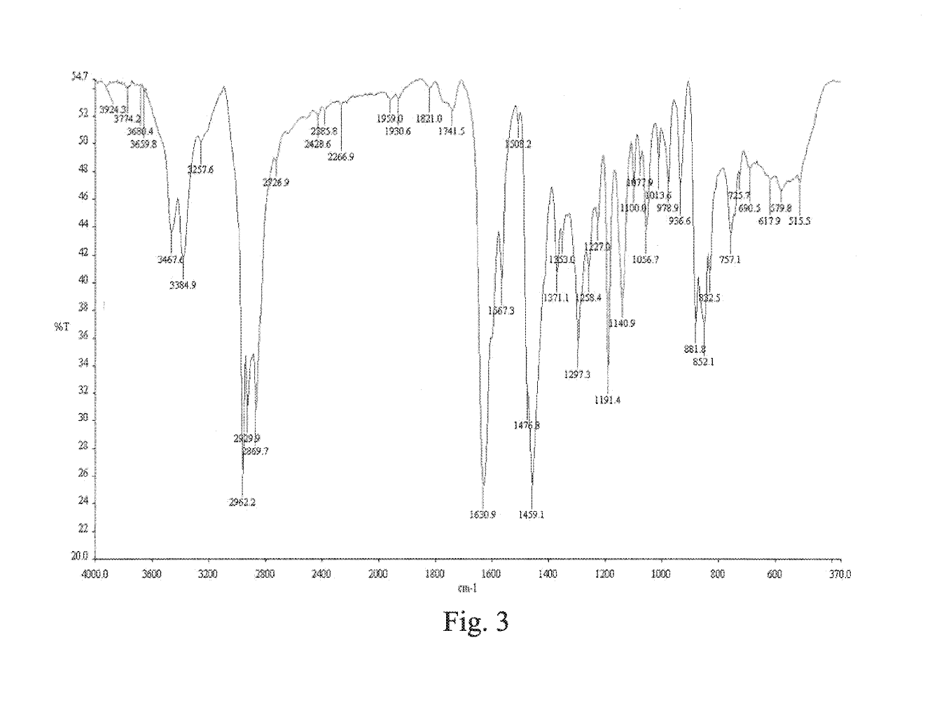

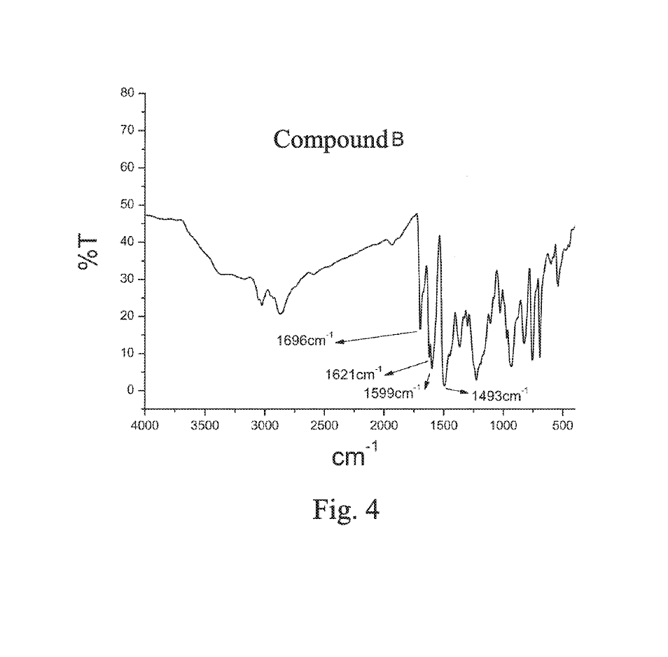

The products afforded in the Production Example were characterized by FTIR (see FIG. 3 and FIG. 4).

FIG. 3 is FTIR spectrum of the reaction precursor Compound A. FIG. 4 is FTIR spectrum of the product Compound B. Two characteristic peaks at 1599 cm⁻¹ and 1493 cm⁻¹ of the benzoxazine compound afforded after the reaction are shown in FIG. 4 but not in FIG. 3, which suggests that Compound B, i.e. the modified benzoxazine has been synthesized. Two characteristic peaks present between 1600 cm⁻¹ and 1700 cm⁻¹ in FIG. 3 and FIG. 4 represent the characteristic peak of —C═N— functional group.

The components for preparing the resin compositions in Examples are listed in Table 1.

### EXAMPLES

The components were thoroughly mixed as per the formulation shown in table 1 to obtain the (uncured) resin composition. In table 1, E1 to E8 represent the Examples of the resin composition of the present disclosure; and C1 to C2 represent the Comparative Examples of the aforesaid resin composition. It should be noted that the examples and comparative examples are provided to further illustrate at least one advantageous effect of several components or the amount thereof The distinguishing between the examples and comparative examples is for the purpose of convenient explanation and is not intended to exclude the comparative examples as part of the invention.

The chemicals used in examples and comparative examples are as follows.

LZ 8280: bisphenol F type benzoxazine resin (BPF-Bz), available from Huntsman;

LZ 8290: bisphenol A type benzoxazine resin (BPA-Bz), available from Huntsman;

LZ 8270: phenolphthalein type benzoxazine resin (phenolphthalein-Bz), available from Huntsman;

BNE-200: bisphenol A novolac epoxy resin, available from ChangChun Plastics;

HP-7200H: dicyclopentadiene type epoxy resin, available from Dainippon Ink and Chemicals, Inc.;

PNE-177: phenolic novolac epoxy resin, available from ChangChun Plastics;

EF-40: styrene-maleic anhydride copolymer, available from Cray Valley;

DDS: diamino diphenyl sulfone, available from Atul LTD.;

HPC-8000: polyester, available from Dainippon Ink and Chemicals, Inc.;

LA-7054: amino triazine novolac (ATN) resin, available from Dainippon Ink and Chemicals, Inc.;

PN: phenolic novolac resin, available from Kolon;

BA-230S: bisphenol A cyanate resin, available from Lonza;

Homide125: bismaleimide, available from HOS-Technik;

SPB-100: phosphazene compound, available from Otsuka Chemical;

SAYTEX 8010: decabromodiphenylethane, available from Albemarle;

XZ92741: DOPO novolac flame retardant, available from Dow Chemical Co.,

2E4MZ: 2-ethyl-4-methylimidazole, available from Shikoku Chemicals Corporation;

525: silica, available from Sibelco.

TABLE 1
The formulations of the resin composition (unit: parts by weight)

| Composition | | | | | | | | | | | | | | | | |
|---|---|---|---|---|---|---|---|---|---|---|---|---|---|---|---|---|
| of the resin | Component | | E1 | C1 | E2 | C2 | E3 | E4 | E5 | E6 | E7 | E8 | E9 | E10 | E11 | C3 |
| oxazine resin | modified Bz | Compound B | 60 | | 50 | | 50 | 50 | 50 | 20 | 25 | 25 | 50 | 30 | 10 | |
| | BPF-Bz | LZ 8280 | | 60 | | | | | | 10 | | | | | | |
| | BPA-Bz | LZ 8290 | | | | 50 | | | | 10 | | | | | | |
| | phenolphthalein-Bz | LZ 8270 | | | | | | | | 10 | | | | | | 50 |
| epoxy resin | bisphenol A | BNE-200 | | | | | | | | | | 50 | | | | |
| | novolac epoxy resin | | | | | | | | | | | | | | | |
| | dicyclopentadiene | HP-7200H | | | 50 | 50 | 50 | 50 | 50 | 50 | 50 | 50 | 70 | 70 | 70 | 50 |
| | type epoxy resin | | | | | | | | | | | | | | | |
| | phenolic novolac | PNE-177 | | | 50 | 50 | 50 | 50 | 50 | 50 | 50 | | 30 | 30 | 30 | 50 |
| | epoxy resin | | | | | | | | | | | | | | | |
| curing agent | styrene-maleic | EF-40 | 30 | 30 | | | | | 30 | | 20 | 20 | | | | |
| | anhydride | | | | | | | | | | | | | | | |
| | copolymer | | | | | | | | | | | | | | | |
| | diamino diphenyl | DDS | | | | | | 6 | | | | | | | | |
| | sulfone | | | | | | | | | | | | | | | |
| | polyester | HPC-8000 | | | | | | | | | 20 | 20 | | | | |
| | ATN | LA-7054 | | | | | 30 | | | | | | | | | 30 |
| | phenolic novolac | PN | | | 10 | 10 | | | | 20 | | | 10 | 10 | 10 | |
| | resin | | | | | | | | | | | | | | | |
| | cyanate resin | BA-230S | | | | | | | | | 5 | 5 | | | | |
| | bismaleimide | Homide125 | | | | | | | | | 15 | 15 | | | | |
| flame | phosphazene | SPB-100 | | | | | | | | | 25 | | | | | |
| retardant | compound | | | | | | | | | | | | | | | |
| | decabromodi- | Saytex 8010 | | | | | | | | | | 50 | | | | |
| | phenylethane | | | | | | | | | | | | | | | |
| | DOPO novolac | XZ92741 | | | | | | | | | 25 | | | | | |
| | flame retardant | | | | | | | | | | | | | | | |
| curing | imidazole | 2E4MZ | 0.3 | 0.3 | 0.3 | 0.3 | 0.3 | 0.3 | 0.3 | 0.3 | 0.3 | 0.3 | 0.3 | 0.3 | 0.3 | 0.3 |
| accelerator | | | | | | | | | | | | | | | | |
| inorganic | silica | 525 | 50 | 50 | 50 | 50 | 50 | 50 | 50 | 50 | 50 | 50 | 50 | 50 | 50 | 50 |
| filler | | | | | | | | | | | | | | | | |
| solvent | MEK | | 60 | 60 | 80 | 80 | 80 | 80 | 80 | 80 | 100 | 100 | 80 | 80 | 80 | 80 |

### Preparation and Analysis of Laminates

After thoroughly mixing in a stirring tank, each of the resin composition examples and comparative examples was placed in an impregnation tank respectively. The prepreg was obtained by impregnating a fiberglass cloth (E-Glass Fabric 2116 type, available from Nan Ya Plastics Corporation) with the resin composition in the impregnation tank followed by baking the impregnated fiberglass cloth into a semi-cured state.

A copper clad laminate was formed by providing four sheets of prepregs prepared from the same batch and two sheets of 18 μm copper foils; stacking the copper foils and the prepregs in the following order from bottom to the top: a copper foil, four sheets of prepregs, and a copper foil; pressing the stacked layers at 210° C. for 2 hours under vacuum condition thereby obtaining a copper clad laminate, wherein the four sheets of prepregs were cured to form an insulating layer between two sheets of copper foils.

The formed copper clad laminates and corresponding copper-free laminates obtained after removal of copper foils were then subjected to physical property tests. The resin content of the copper foil-free laminates (i.e. laminates made from four sheets of prepregs) was about 55%. The physical properties under copper foil-free condition were measured by using the copper-free laminates made from four sheets of prepregs except dielectric constant and dissipation factor under copper foil-free condition, which were measured by using the copper-free laminates made from two sheets of prepregs. The physical property tested include: glass transition temperature (Tg, measured by DSC apparatus according to the method described in IPC-TM-650 2.4.25c); heat resistance (T288, measured by thermomechanical analyzer (TMA); to measure the time for a copper-containing laminate not to get de-laminated under 288 degree C. according to the method described in IPC-TM-650 2.4.24.1); dielectric constant (Dk, measured by Microwave Dielectrometer (AET) at frequency of 10 GHz according to the method described in JIS C2565; the lower the Dk value, the better the dielectric property; it is recognized in the art that when the difference of the Dk value between an example and a comparative example is equal to or greater than 0.1, such a difference is significant); dissipation factor (Df, measured by Microwave Dielectrometer (AET) at frequency of 10 GHz according to the method described in JIS C2565; the lower the Df value, the better the dielectric property; it is recognized in the art that when the difference of the Df value between an example and a comparative example is equal to or greater than 0.001, such a difference is significant); flaming test (according the the method in UL94 standard, where classification V-0 is better than V-1 and V-1 is better than V-2). The results of the tests are listed in Table 2.

TABLE 2
Evaluation of properties of the laminates

| Laminate property | Parameter | Unit | E1 | C1 | E2 | C2 | E3 | E4 | E5 |
|---|---|---|---|---|---|---|---|---|---|
| Tg | DSC | ° C. | 200 | 150 | 205 | 161 | 200 | 240 | 205 |
| Heat resistance | T288 (TMA) | min | N/D | N/D | >30 | 15 | >30 | >30 | >30 |
| Dielectric constant | Dk@10 GHz | N/A | 3.85 | 3.90 | 3.95 | 4.00 | 3.95 | 3.98 | 3.95 |
| Dissipation factor | Df@10 GHz | N/A | 0.0070 | 0.0130 | 0.0078 | 0.0135 | 0.0082 | 0.0085 | 0.0072 |
| Flame retardancy | UL94 | Rating | V-2 | V-2 | V-2 | V-2 | V-2 | V-2 | V-2 |
| Laminate property | Parameter | Unit | E6 | E7 | E8 | E9 | E10 | E11 | C3 |
| Tg | DSC | ° C. | 220 | 210 | 212 | 201 | 189 | 168 | 203 |
| Heat resistance | T288 (TMA) | min | >30 | >30 | >30 | >30 | >30 | >30 | >30 |
| Dielectric constant | Dk@10 GHz | N/A | 3.98 | 3.85 | 3.81 | 3.95 | 3.98 | 4.05 | 4.13 |
| Dissipation factor | Df@10 GHz | N/A | 0.0085 | 0.0065 | 0.0062 | 0.0078 | 0.0093 | 0.0105 | 0.0151 |
| Flame retardancy | UL94 | Rating | V-2 | V-0 | V-0 | V-2 | V-2 | V-2 | V-1 |

Note 1:
In table 2, Dk@10 GHz represents that the result is obtained by measuring at 10 GHz by using the method described in JIS C2565; Df@10 GHz represents that the result is obtained by measuring at 10 GHz by using the method described in JIS C2565.
Note 2:
It is shown in table 2 that the resin composition E2 exhibits Df value of 0.0043 at 1 GHz (Df@1 GHz = 0.0043), 0.0067 at 6 GHz (Df@6 GHz = 0.0067), and 0.0078 at 10 GHz (Df@10 GHz = 0.0078).

From the data of E1-E2 and C1-C2 shown in tables 1 and 2, it can be found that the laminates made from modified Bz-containing composition had significantly higher glass transition temperature (Tg) and significantly better (i.e. lower) dissipation factor (Df) when compared to the laminates having BPF-Bz or BPA-Bz added.

E3 to E5 were prepared in order to determine the influence of the change in parameters (different types or amounts of co-curing agents) on the characteristics of the laminates. From E6, it can be found that the combination of the modified Bz composition with other types of Bz composition could also achieve excellent general characteristics of the laminates, such as higher glass transition temperature (Tg) and better dissipation factor (Df). Further, from the data of E7 and E8, it can be found that better flame retardancy effect (classification V-0) can be achieved by adding a flame retardant. Given above, the aforesaid modified Bz may be combined with other components in different amount to adjust various characteristics of the laminate, thereby satisfying the demands in practice.

Compared examples with comparative examples, it can be found that the laminate made from C1 or C2 composition respectively having conventional BPA type benzoxazine compound and BPF type benzoxazine compound added, has poor (lower) glass transition temperature and poor (higher) dissipation factor. Regarding the laminate made from C3 composition having phenolphthalein type benzoxazine compound, although higher glass transition temperature can be achieved, dissipation factor is the worst among examples and comparative examples. As shown in the comparison, the laminate made from the modified benzoxazine compound of the present disclosure can have not only better (higher) glass transition temperature but also better (lower) dissipation factor.

As stated above, the resin composition of the present disclosure comprising defined components and the ratio among thereof may achieve low dielectric constant, low dissipation factor, high heat resistance, and high flame retardency. The resin composition is useful in preparing the prepreg or the resin film for production of the laminate (copper clad laminate) and printed circuit board. In terms of industrial applicability, the products derived from the present disclosure may satisfy the current market needs.

While this invention has been disclosed in this patent application by reference to the details of preferred embodiments of the invention, it is to be understood that this disclosure is intended in an illustrative rather than in a limiting sense, as it is contemplated that modifications will readily occur to those skilled in the art, within the spirit of the invention and the scope of the appended claims.

1. A modified oxazine compound having a structure represented by the following formula (1) or formula (2): 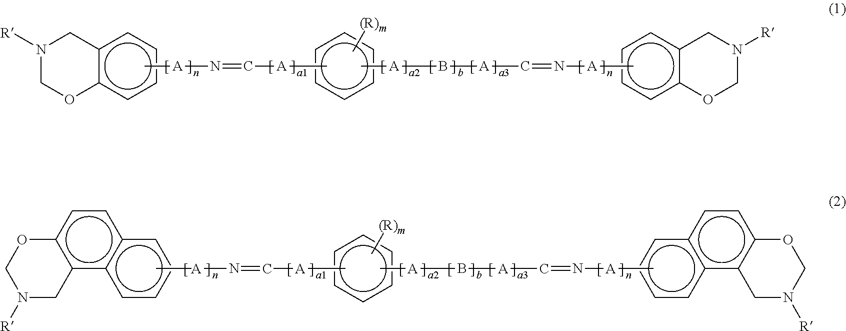
wherein
R is an aliphatic hydrocarbyl group or an aryl group;
R′ is selected from the group consisting of imino, allyl, a C₁-C₂₀ aliphatic hydrocarbyl group, dicyclopentadienyl, and an aryl group;
A is selected from the group consisting of —CH₂—, —CH(CH₃)—, and —C(CH₃)₂— and is the same or different at each occurrence;
B is an arylene group;
m is 0 to 4;
n is 0 to 1; and
a1, a2, a3 and b are each independently 0 or 1.

2. The modified benzoxazine compound as claimed in claim 1, which is selected from the group consisting of formula (6), formula (7), formula (8), formula (9), and formula (10):

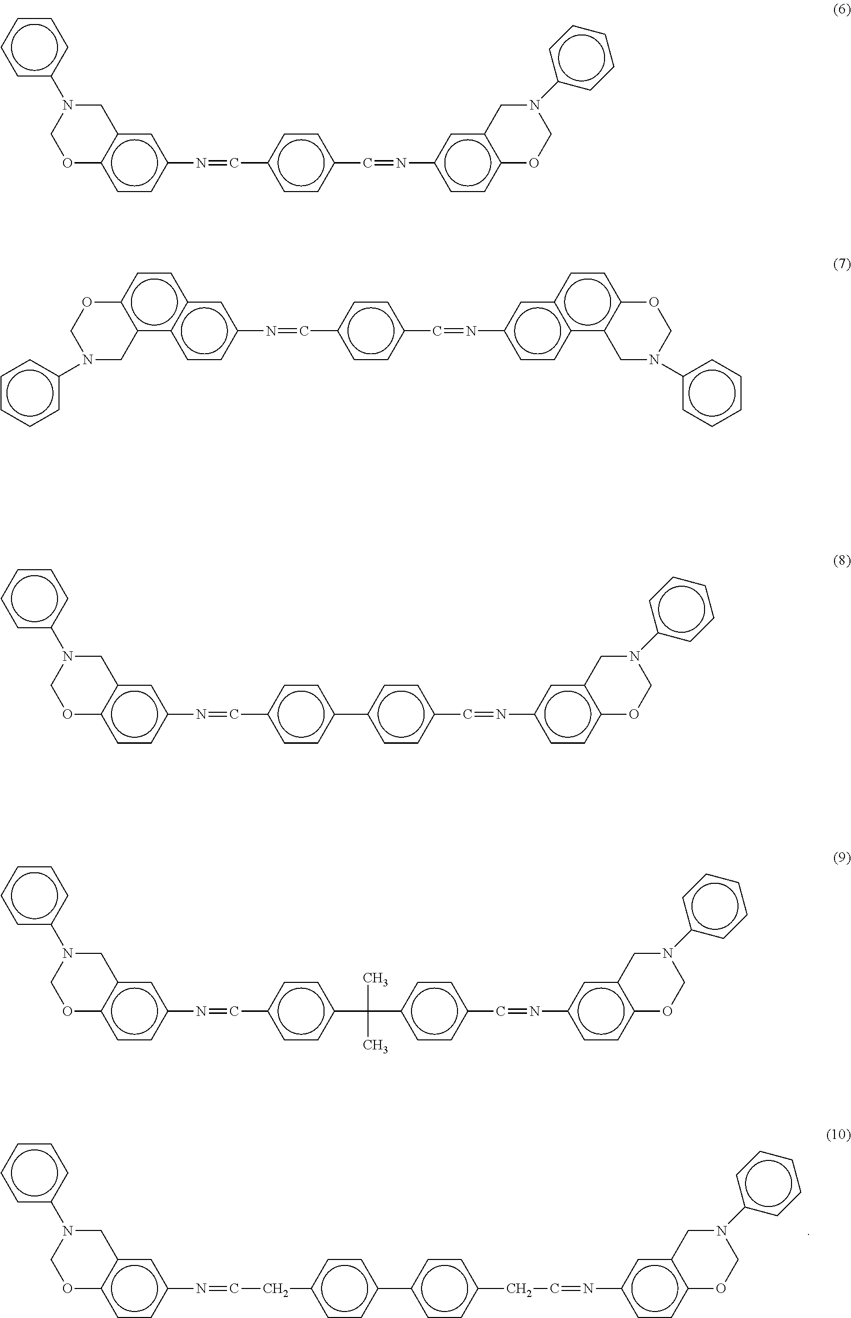

3. A method of producing a modified benzoxazine compound comprising: reacting a phthalaldehyde compound with an aminophenol compound in a solvent to form an azomethine group-containing phenol compound; and reacting the azomethine group-containing phenol compound with a primary amine and formaldehyde.

4. The method as claimed in claim 3, wherein the phthalaldehyde compound is one represented by formula (3): 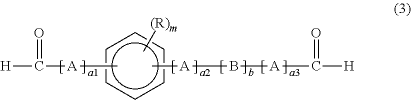
wherein
R is an aliphatic hydrocarbyl group or an aryl group;
A is selected from the group consisting of —CH₂—, —CH(CH₃)—, and —C(CH₃)₂— and is the same or different at each occurrence;
B is an arylene group;
m is 0 to 4; and
a1, a2, a3 and b are each independently 0 or 1.

5. The method as claimed in claim 3, wherein the aminophenol compound is one represented by formula (4) or formula (5): 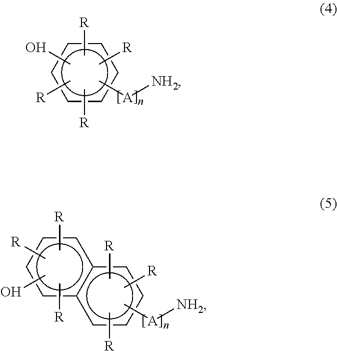
wherein
R are each independently hydrogen, an aliphatic hydrocarbyl group or an aryl group;
A is selected from the group consisting of —CH₂—, —CH(CH₃)—, —C(CH₃)₂—, an aliphatic hydrocarbylene group, and an arylene group; and
n is 0 or 1.

6. The method as claimed in claim 3, wherein the primary amine is one having a general formula: R′NH₂, wherein R′ is selected from the group consisting of imino, allyl, a C₁-C₂₀ aliphatic hydrocarbyl group, dicyclopentadienyl, and an aryl group.

7. A resin composition, comprising: (A) a modified oxazine compound according to claim 1 or a prepolymer thereof or a mixture thereof; and (B) a crosslinking agent.

8. The resin composition as claimed in claim 7, wherein the crosslinking agent (B) is selected from the group consisting of an epoxy resin, a cyanate resin, isocyanate, a polyphenylene oxide resin, maleimide, a polyamide, a polyimide, a phenoxy resin, a styrene-maleic anhydride copolymer, a polyester, a polyolefin, a phenol resin, an amine-based curing agent, an anhydride-based curing agent, diallyl bisphenol A, and a combination thereof.

9. The resin composition as claimed in claim 7, further comprising a modifier selected from the group consisting of flame retardant, curing accelerator, inorganic filler, surfactant, solvent, and toughening agent.

10. An article made from the resin composition according to claims 7, wherein the article is a resin film, a prepreg, a laminate or a printed circuit board.
